(19) United States
(12) Patent Application Publication
(10) Pub. No.: US 2020/0376157 A1
(43) Pub. Date: Dec. 3, 2020

(54) HAEMOSTATIC MATERIAL

(71) Applicant: MEDTRADE PRODUCTS LIMITED, Crewe, Cheshire (GB)

(72) Inventors: Craig HARDY, Cardigan, Pembrokeshire (GB); Andrew HOGGARTH, Crewe, Cheshire (GB); Matthew GRIST, Nantwich, Cheshire (GB)

(73) Assignee: MEDTRADE PRODUCTS LIMITED, Crewe, Cheshire (GB)

(21) Appl. No.: 16/968,710

(22) PCT Filed: Feb. 14, 2019
(86) PCT No.: PCT/GB2019/050396

§ 371 Date: Aug. 10, 2020

(30) Foreign Application Priority Data
Feb. 14, 2018 (GB) 1802380.4

(51) Int. Cl. A61L 15/44; A61K 31/195; A61K 31/722; A61K 31/78; A61L 15/22; A61L 15/58

(52) U.S. Cl. CPC A61L 15/44; A61K 31/195; A61K 31/722; A61K 31/78; A61L 2400/04; A61L 15/58; A61L 2300/214; A61L 2300/252; A61L 2300/418; A61L 15/22

(57) ABSTRACT

A haemostatic material that is effective at controlling the flow of blood from both standard and coagulopathic wound injuries that maintains a reduced compression time minimising the requirement for resuscitation fluids, and being easy and safe to use.

The present invention relates to a haemostatic material for use in controlling bleeding.

There are many circumstances in which animals, both human and non-human, may become injured or wounded causing bleeding. In the case of minor wounds, the bleeding may be stemmed by the natural haemostatic mechanisms of the body which lead to coagulation of the blood to form solid clots which prevent haemorrhage and aid repair of damaged blood vessels.

Traditionally the primary technique adopted for stemming blood flow from a wound is the application of continuous pressure to the wound. This enables clotting factors to collect at the wound site and form a congealed blood mass to stem blood flow. However, this technique is not suitable for severe wounds and wounds having multiple bleeding points. Therefore, bleeding out continues to be a major cause of death.

Death caused by bleeding out is a particular problem on the battlefield. Typically, wounds arising in this situation are accompanied by significant bleeding, and many result in death. Bleeding out is also a significant cause of death amongst the civilian population following trauma.

Attempts have been made to provide products which facilitate the stemming of blood flow from a wound. These include a product sold under the brand name Quick-clot®. Simplistically, this product contains a carrier material which is coated with an active compound, which, when applied to the wound with pressure, is able to stem the blood flow.

More specifically, Quick-clot® comprises a zeolite compound which absorbs water from the blood flowing from a wound, such that the clotting factors present in the blood become concentrated and the blood coagulates more quickly, thereby the zeolite and the coagulated blood together form a coagulum to stem blood flow.

Whilst effective, these compositions are not without problems, as they require continuous pressure to control the bleeding. The guidance provided by the Tactical Combat Casualty Care (TCCC) in November 2009 indicated that a minimum of three minutes' compression should be applied when using a haemostatic bandage, specifically Combat Gauze®. Other examples of haemostatic products requiring a minimum of three minutes' compression include, but are not limited to, Celox® Gauze (Medtrade Products Ltd) and Chitogauze® (Hemcon).

More recently, as described in patent US 2014/105950, bioadhesive agents have been used and incorporated into the above described haemostatic dressings to reduce compression times, potentially reducing blood loss and overall treatment time.

A further aspect to this work that has been highlighted by medics is the impairment of the body's ability to control bleeding due to coagulopathy. Coagulopathy may be defined as a condition in which the blood's ability to coagulate (form clots) is impaired. This condition can cause a tendency towards prolonged or excessive bleeding, which may occur following injury or medical procedures. The resulting effect on treatment using the above described haemostatic products is an extension to the time required for pressure, i.e. requiring further prolonged compression periods compared to non-coagulopathic persons.

In situations where the person is coagulopathic, this may result in prolonged bleeding after treatment which would require further medical intervention prior to the surgical hospital treatment (field or civilian). Increased treatment times to obtain haemostasis in coagulopathic persons may also result in further endangering the medic's life when treatment under fire or may result in delayed response to other casualties or injuries.

A further aspect to bleeding injuries is the requirement for fluid and resuscitation fluids to be administered.

Testing undertaken by the Institute for Surgical Research in the USA has reported a lack of haemostasis for several existing products using an in-vivo coagulopathic model or prolonged compression for products containing bioadhesives.

It is therefore an object of the present invention to provide a haemostatic material which is effective at controlling the flow of blood from both standard and coagulopathic wound injuries, whilst maintaining a reduced compression time, minimising the requirement for resuscitation fluids, and being easy and safe to use.

Therefore, according to a first aspect of the present invention, there is provided a haemostatic composition comprising a haemostat agent, a bioadhesive agent and an antifibrinolytic agent or derivative thereof.

The composition of the invention may be in several forms, comprising but not limited to, granules, powders, flakes, foams, solutions, or gels, for which these may be applied directly to the wound or coated, carried, or delivered on a carrier material.

By “haemostat agent”, it is meant herein a substance that promotes haemostasis. The haemostat agent may be capable of producing a clot or plug to stop or reduce bleeding when brought into contact with blood.

A physiological target site for the haemostatic material may be any site in or on the body of an animal. The animal may be a human or a non-human animal. The physiological target site may be a wound or it may be an opening in a body caused during a medical procedure, for example during surgery. Hereinafter, the physiological target site is referred to as a wound for convenience and illustrative purposes only.

Beneficially, the haemostatic material of the present invention can be applied by a person with only basic medical training. It is a matter of simply applying the material to the physiological target site followed by pressure.

Further still, the haemostatic material is easy to handle and apply. It is typically stored dry prior to application.

Products which take advantage of biological processes tend to be temperature dependent. Often, patients suffering blood loss are either very hot due to exertions on the battlefield, or very cold as they have been exposed to cold conditions. Currently available products are less effective at such temperature extremes. Advantageously, the material of the present invention is substantially unaffected by temperature fluctuations and therefore works equally well at temperatures both above and below normal body temperatures. By “normal body temperature” it is meant about 37° C.

The haemostatic composition of the present invention is capable of effectively controlling bleeding with a reduced treatment period compared to the TCCC guidance of a minimum of three minutes' compression post packing using a haemostatic bandage in both normal and coagulopathic conditions. Advantageously, this results in a subject being stabilised in a shorter time period before deployment to a medical area. By ‘treatment’, it is meant the time taken to pack and fill the wound or incision with a haemostat composition, which involves the compression of the site of the bleeding.

The present invention is able to control bleeding effectively with about 45 seconds of treatment, compared to the at least three minutes indicated in the TCCC guidance.

The haemostat agent may be any material with haemostatic properties. The haemostat agent may comprise a polymer containing one or more glucosamine units therein. Examples of haemostat agents include, but are not limited to, oxidised regenerated cellulose, kaolin, gelatin, calcium ions, zeolite, collagen, chitin, chitosan or a chitosan salt, derivatives of chitosan, derivatives of chitin, and any combination thereof. Glucosamine is of course a part of the structure of chitosan and chitin. The haemostat agent is preferably a chitosan salt.

The term ‘derivative’ is used herein to refer to a compound that is derived from chitosan or chitin following one or more chemical reactions or modifications. The one or more chemical reactions or modifications may involve substitution of one or more of the amino or hydroxyl protons in chitosan or chitin; or partial deacetylation of chitin. For example, a chitin derivative may include a partially deacetylated chitin, which may have different percentages of deacetylation, as desired. Typically, the partially deacetylated chitin suitable for use in the present invention has a deacetylation degree above about 50%, more typically above about 75% and most typically above about 85%. Also included within the terms ‘chitosan or chitin derivatives’ are reaction products of chitosan or chitin with other compounds. Such reaction products include, but are not limited to, carboxymethyl chitosan, hydroxyl butyl chitin, N-acyl chitosan, O-acyl chitosan, N-alkyl chitosan, O-alkyl chitosan, N-alkylidene chitosan, O-sulfonyl chitosan, sulphated chitosan, phosphorylated chitosan, nitrated chitosan, alkalichitin, alkalichitosan, or metal chelates with chitosan, etc.

Chitosan is a derivative of solid waste from shell fish processing and can be extracted from fungus culture. It is a water insoluble polymeric material. Therefore, chitosan for use with the present invention is first converted into a water-soluble salt. The chitosan salt is soluble in blood to form a gel which sterns blood flow.

Chitosan salts are ideally suited for the applications described herein as chitosan is readily broken down in the body. Chitosan is converted to glucosamine by the enzyme lysozyme and is therefore excreted from the body naturally. It is not necessary to take any measures to remove the chitosan from the body.

Furthermore, chitosan salts exhibit mild antibacterial properties and as such their use reduces the risk of infection.

Exemplary chitosan salts which are suitable for use with the present invention include, but are not limited to, any of the following either alone or in combination: acetate, lactate, succinate, malate, sulphate or acrylate. They are typically in powder form.

Good results have been observed wherein the chitosan salt comprises, or is, chitosan lactate.

The chitosan salt is prepared by combining chitosan with an appropriate acid. It will be appreciated that the acid may be any inorganic or organic acid which yields a chitosan salt which is soluble under the conditions associated with a human or animal body, particularly in blood. Suitable acids would be recognised by a skilled person. For example, chitosan phosphate is insoluble in such conditions and so phosphoric acid is unsuitable.

The haemostat agent may constitute at least 20% by weight of the haemostatic material, or more typically at least about 80% by weight. Typically, the haemostat agent constitutes from 20-99% by weight of the haemostatic material, preferably from 45-95% by weight of the haemostatic material.

The haemostat agent is typically granular, but may comprise short fibres, sponges, fabrics, films, powders, liquid, gels or liquid coating. The short fibres may be no more than about 7.5 mm in length, more typically no more than about 5 mm in length.

The haemostat agent typically has a pH of from about 3.5 to about 8.0. The pH is largely dependent upon the particular haemostat agent used, as they each have a different pH.

By “bioadhesive agent”, it is meant a natural or synthetic biocompatible substance that binds to a biological substrate. The biological substrate may be, for example, moist tissue at a wound site. In effect, a bioadhesive agent may promote adhesion between two materials, one of which is biological in nature, such that the materials are held together for an extended period of time. The bioadhesive agent typically exhibits low adhesion to dry surfaces, for example gloves or intact skin, and high adhesion to wet/moist surfaces, for example wounds or internal organs. Consequently, the haemostatic material comprising the bioadhesive agent and the haemostat agent should preferably exhibit low adhesion to dry surfaces and high adhesion to wet/moist surfaces. Preferably, the haemostatic material exhibits no adhesion to dry surfaces. Beneficially, this property of the bioadhesive agent provides a haemostatic material that is both easy to handle and enables the haemostatic material to effectively control bleeding within a reduced compression period compared to the TCCC guidance of a minimum of three minutes compression.

The bioadhesive agent should preferably be compatible with the haemostat agent and not interfere with the efficacy of the haemostatic material. The bioadhesive agent is typically a solid, dry, material.

By ‘low adhesion’, it is meant adhesion to a surface with a peel force of 0.05 N per 25 mm of material (which is denoted as 0.05N/25 mm) or below. No adhesion is effectively measured as 0.0 N/25 mm.

By ‘high adhesion’, it is meant adhesion to a surface with a peel force of 0.25 N/25 mm or above. Preferably, the adhesion to a wet/moist surface exhibits a peel force of 0.7 N/25 mm or above and more preferably 1.0 N/25 mm or above. The adhesion to a wet/moist surface typically exhibits a peel force in the range 0.6-2.0 N/25 mm.

Thus, the bioadhesive agent may promote the adhesion of the haemostatic agent to moist tissue at the wound site. Beneficially, this allows the compression time required for clotting to be reduced without the blood pressure forcing the haemostatic agent from the wound site.

The bioadhesive agent may constitute up to 90% by weight of the haemostatic material. Preferably, the bioadhesive agent may constitute up to 20% by weight of the haemostatic material, more preferably from 2 to 20% by weight of the haemostatic material, even more preferably from 5 to 10% by weight of the haemostatic material and most preferably from 7 to 8% by weight of the haemostatic material. At these preferred ranges, the bioadhesive agent is optimised for adhesion to the wet or moist tissue without causing adverse effects upon removal, such as for example wound re-opening.

The bioadhesive agent should be a material which generates a high adhesion when applied to wet/moist substrates. The bioadhesive agent may be selected from any of the following either alone or in combination: a carbomer, polyvinyl alcohol (PVA), polyvinylpyrrolidone (PVP), 2-acrylamido-2-methylpropane sulfonic acid, or a high molecular weight acrylic acid polymer cross-linked with divinyl glycol or the salts of polyacrylic acid cross-linked with divinyl glycol. Preferably, the bioadhesive agent comprises high molecular weight cross-linked polymers of acrylic acid. By ‘high molecular weight’ it is meant a molecular weight of at least 50,000 g/mol. Preferably, the molecular weight is at least 60,000 g/mol and more preferably from 100,000 to 300,000 g/mol. In such embodiments, the bioadhesive agent may be a homopolymer comprising a polymer of acrylic acid cross-linked with allyl sucrose or allyl pentaerythritol; a copolymer comprising a polymer of acrylic acid and C₁₀-C₃₀ alkyl acrylate cross-linked with allyl pentaerythritol; a carbomer homopolymer or a copolymer comprising a block copolymer of polyethylene glycol and a long chain alkyl acid ester; or mixtures thereof. Examples of such polymers include Carbopol® NF934, NF974, NF971 and NF980.

The bioadhesive agent provides the composition of the present invention with excellent wet stick properties. By “wet stick” it is meant adhesion to wet or moist tissue. This allows for the bioadhesive agent to promote adhesion between the haemostat agent and moist tissue at the wound site.

In some embodiments, the haemostat agent and the bioadhesive agent are typically present in a ratio of at least 3:1. Typically, the haemostat agent and bioadhesive agent are present in a ratio of at least 4:1 and more preferably in a ratio of at least 9:1.

By “antifibrinolytic agent”, it is meant a natural or synthetic substance which inhibits fibrinolysis. Fibrinolysis is a process that prevents blood clots from growing. This process has two types: primary fibrinolysis and secondary fibrinolysis. The primary type is a normal body process, whereas secondary fibrinolysis is the breakdown of clots due to a medicine, a medical disorder, or some other cause. Therefore, antifibrinolytic agents prevents the breakdown of blood clots, which should be stronger and last longer than if the antifibrinolytic agent was not present.

The antifibrinolytic agent may be chemically bonded, salted or associated with the haemostatic agent, or it may be independent of the haemostatic and bioadhesive agents.

The antifibrinolytic agent may comprise a plasminogen activator inhibitor, such as a serine protease inhibitor. Non-limiting examples of such serine protease inhibitors include plasminogen activator inhibitor-1 (PAL-1), which is also known as endothelial plasminogen activator inhibitor or serpin E1, or aprotinin. PAI-1 is a serine protease inhibitor that functions as the principal inhibitor of tissue plasminogen activator (WA) and urokinase (uPA), the activators of plasminogen and hence fibrinolysis. Aprotinin is a competitive inhibitor of several serine proteases, specifically trypsin, chymotrypsin and plasmin at a concentration of about 125,000 and kallikrein at 300,000 IU/ml. Its action on kallikrein leads to the inhibition of the formation of factor XIIa. As a result, both the intrinsic pathway of coagulation and fibrinolysis are inhibited. Its action on plasmin independently slows fibrinolysis.

Alternatively, the antifibrinolytic agent may comprise a glycoprotein, such as fibrinogen; or tranexamic acid.

Alternatively, the antifibrinolytic agent may comprise a C2-C12 aminocarboxylic acid, a C4-C8 aminocarboxylic acid, or a C5-C7 aminocarboxylic acid, such as a C6 aminocarboxylic acid, e.g. aminocaproic acid or epsilon-aminocaproic acid.

Alternatively, the antifibrinolytic agent may comprise an aminobenzoic acid, such as aminomethylbenzoic acid.

Any one or more of these antifibrinolytic agents, or derivatives thereof, may be used either alone or in combination.

The term ‘derivative’ in relation to the antifibrinolytic agents is used herein to refer to any compounds which are directly derived or derivable from any of the above-listed compounds and which also exhibit antifibrinolytic behaviour.

The antifibrinolytic agent is typically present in an amount of about 0.01 to about 99.9 wt % of the haemostat composition; more typically from about 0.1 to about 90 wt %, more typically from about 1 to about 80 wt %, more typically from about 2 to about 70 wt %, more typically from about 5 to about 60 wt %, more typically from about 10 to about 50 wt %, more typically from about 12 to about 40 wt %, more typically from about 15 to about 35 wt %, more typically from about 20 to about 30 wt %, more typically from about 22 to about 28 wt %, such as about 25 wt %.

The haemostat agent may further comprise an inert material. By “inert” it is meant a material having non-haemostatic or poorly haemostatic properties and having low adhesion to wet/moist surfaces; i.e. a material which alone does not exhibit any significant haemostasis within a period of about three minutes, five minutes, or even within ten minutes upon application to a bleeding site.

Exemplary inert materials include but are not limited to non-haemostatic cellulose, non-haemostatic sand, non-haemostatic clay, non-haemostatic alginate, microcrystalline cellulose, guar gum, xanthan gum, non-haemostatic chitosan, non-haemostatic chitin, dextran, sucrose, lactose, pectin, carboxymethylcellulose, hydroethyl cellulose, ground corn meal, polyacrylic acid, barium sulphate, starch, or combinations of any two or more thereof. Typically, one or more inert materials selected from non-haemostatic chitosan, non-haemostatic chitin and carboxymethylcellulose are used.

The inert material may be added to the haemostat agent in an amount up to about 95% by weight of the total composition, typically up to about 80% by weight, and more typically up to about 50% by weight. The inert material is typically blended with the haemostat agent, but may be dispersed in solution with the haemostat agent and dried.

Typically, the inert material is granular, but can be in the form of a powder, foam, fibres, or films.

The haemostat agent may further comprise a medical surfactant. By “medical surfactant” it is meant any surfactant that is pharmaceutically acceptable for contact with or administration to a human or animal body and does not cause any significant detrimental effects to the human or animal body. Exemplary medical surfactants for use in the present invention include any of the following either alone or in combination: block copolymers based on ethylene oxide and propylene oxide (e.g. BASF Pluronics®), glycerol, polyethylene glycol, propylene glycol, fatty acids such as lauric acid, oleic acid, other fatty acids and fatty acid salts, silicone-based surfactants and emulsifiers. Typically, the medical surfactants include lauric acid and oleic acid.

The medical surfactant may typically constitute from about 0.001 to about 10% by weight of the haemostat agent.

More advantageously, the medical surfactant constitutes from about 0.5 to about 1% by weight of the haemostat agent. Advantageously, the presence of a surfactant gives rise to excellent wetting out properties. The way in which the haemostat agent wets out is important to its performance That is, the haemostat agent can absorb the blood too quickly and simply mix with the blood without sufficient gelation having occurred to form a gel clot which is capable of stemming blood flow. On the other hand, if the haemostat agent absorbs the blood too slowly gelation occurs in only a small amount of the haemostat agent, generally the first few millimetres depth of the haemostat agent closest to the wound site. In this case the gel clot which forms is not sufficiently dense to stem the blood flow for a sufficient period of time to allow the patient to be moved to a medical centre. Typically, such a gel clot will break up as the patient is moved and bleeding will resume.

It has been found that by adding an amount of an inert material and/or an amount of a medical surfactant to the haemostat agent, i.e. in effect diluting the quantity of haemostat agent, the performance of the haemostat agent is actually enhanced further. A combination of the inert material and the medical surfactant together is particularly advantageous as the presence of the inert material further enhances the properties of the medical surfactant, and vice versa.

The particle size of the haemostat agent can affect the performance of the haemostatic material of the present invention. The particle size is measured by the size of sieve through which the particle will pass or be retained by.

For example, when the haemostat agent is in particulate or granular form, it may have an average particle size of greater than about 200 mesh such that it will not pass through a 200 mesh sieve. The average particle size may typically be greater than about 100 mesh, still more typically greater than about 50 mesh, and it is not desired that the particles or granules are able to pass through a 40 mesh sieve.

More advantageously, the particle size of the inert material will be substantially equivalent to that of the haemostat agent. By “substantially equivalent” it is meant that the relative sizes of the particles do not differ by more than about 25%, more typically by more than about 10%. The optimum particle size is achieved by grinding the haemostat agent and sorting by any suitable means such as sieving. Such sizing processes are well known to those skilled in the art and will not be described further.

The haemostatic composition may be administered to the wound in any particular form, such as for example, a dry powder, solution, foam or gel.

The haemostatic composition may be applied to a carrier material for application to the wound site. The carrier material may comprise a woven material, or a viscose non-woven material, or alternatively it may comprise a thin flexible substrate, a woven gauze, a film, a foam, a solution, or a sheet gel. The composition of the invention may also be in a freeze-dried format.

The composition may or may not be degradable in conditions associated with wounds in or on a human or animal body. In one example, the material of the carrier material may be safely degradable in the body within a reasonable period of time, such as about 30 days, so that the whole haemostatic material piece can be left in place after surgical use or treatment. Examples of safe and degradable materials for use in the composition include, but are not limited to, oxidised cellulose, gelatin, dextran, collagen, polycaprylactone, polylactide acid, polylactide-co-glycolide, polyglycolide, chitin, etc.

The haemostat agent may be applied to the carrier material by a variety of methods. These include bonding the haemostat agent to the carrier material using an adhesive; applying a solution containing the haemostat agent to the carrier material, coating the carrier material and drying the solution; or by heat bonding. The haemostat agent may also be incorporated into the carrier material during the processing of the carrier materials.

The composition may take any suitable form and may be provided in a range of different sizes, shapes and thicknesses necessary to deal with a wound, such as square, rectangular, circular or elliptical. For example, the material may be a generally flat shape with little height relative to its width/depth. Any regular or irregular shape may be employed. It may be provided in large sheets which can be cut to the required size.

The haemostatic composition may be provided in a sterile or non-sterile form. Where the material is provided in a sterile form, sterilisation may be carried out using any of the conventionally known methods, such as gamma irradiation, electron beam treatment, heat treatment, ethylene oxide (EtO) sterilization etc. A material in a non-sterile form may be provided in combination with one or more preservatives or antimicrobial agent, such as silver and its salts.

When the haemostatic composition is sterilised using ethylene oxide, this may comprise exposing the intermediate device to gaseous ethylene oxide. The sterilisation phase may be conducted in a chamber, which is preferably sealed.

According to a further aspect of the present invention, there is provided a method of haemostasis, the method comprising the steps of applying the haemostatic composition comprising a haemostat agent, a bioadhesive agent and an antifibrinolytic agent or derivative thereof, as described herein, to a physiological target site; and applying pressure to the haemostatic material for a period of less than about one minute, or for less than about 55 seconds, or for less than about 50 seconds, or for less than about 45 seconds.

According to a further aspect of the present invention, there is provided a haemostatic composition comprising a haemostat agent, a bioadhesive agent and an antifibrinolytic agent or derivative thereof, for use in stemming blood flow from a physiological target site.

The pressure may be applied to the target site for a period of between about 30 seconds to one minute. In some embodiments, the pressure may be applied to the wound site for between about 35 seconds to about 55 seconds; or between about 40 seconds to about 50 seconds; or for about 45 seconds. An advantage of the present invention is the quick time taken to sufficiently clot blood flowing from a wound site. Thus, sufficient clotting forms within about one minute such that the pressure may be applied to the target site for a shorter time to obtain the desired effect. In some embodiments, the pressure may be applied to the wound site for less than about 55 seconds to have the desired effect, and preferably less than about 50 seconds.

According to a further aspect of the present invention, there is provided a carrier material comprising a haemostatic composition comprising a haemostat agent, a bioadhesive agent and an antifibrinolytic agent or derivative thereof, applied to the carrier material.

The carrier material may comprise any of the features of the carrier material described hereinbefore. Preferably, the carrier material comprises a viscose gauze.

According to a further aspect of the present invention, there is provided a method of manufacturing a haemostatic composition comprising a haemostat agent, a bioadhesive agent and an antifibrinolytic agent or derivative thereof, the method comprising the step of combining a haemostat agent with a bioadhesive agent and an antifibrinolytic agent or derivative thereof.

Preferably, the method of manufacturing the haemostatic material comprises the steps of: (1) dispensing a pre-determined weight of a haemostat agent and optionally an inert material into a mixing vessel; (2) dispensing a pre-determined weight of a bioadhesive agent into the mixing vessel containing the haemostat and optional inert material; (3) dispensing a pre-determined weight of an antifibrinolytic agent or derivative thereof; and (4) mixing the haemostat agent, bioadhesive agent and antifibrinolytic agent or derivative thereof.

The invention will now be further described with reference to the following Example, which is intended to be illustrative only, and non-limiting upon the scope of the invention.

### EXAMPLE 1

A 7 wt % bioadhesive agent (high molecular weight cross-linked polymers of acrylic acid (Carbopol® 980NF)) and was blended with a chitosan derivatives/non-haemostatic chitosan blend. The chitosan derivatives consisted of chitosan lactate and chitosan tranexamate, whereby the chitosan was salted using combinations of chitosan, lactic acid and tranexamic acid. The mixture was double-coated onto viscose gauze at a coat weight of 45 gsm. This provided a haemostatic composition according to the invention.

### In Vivo

To confirm that the invention exhibits real advantages in compression time, and provides evidence of efficacy with a total packing and compression time of 45 seconds, the composition of Example 1 was tested in a porcine model using a 6 mm femoral artery sever model as per the ISR model in both normal and coagulopathic conditions.

For normal conditions, a 6 mm sever was surgically made to the femoral artery of a porcine model. The artery was allowed to bleed out for a period of 45 seconds, following which the haemostatic material was applied to the bleed site, utilising a total combined packing and compression period of 45 seconds. Following the compression period, the wound was assessed for bleeding. If bleeding re-occurred, the haemostatic material compressed for a further one minute's pressure. Any re-bleeding after this point was classified as a fail.

For coagulopathic conditions, 25% of pig's blood volume was replaced with Hextend fluid (25% hemodilution) and hypothermia (core temperature 34°-35° C.) was induced in the swine prior to arterial injury and hemorrhage. A 6 mm sever was surgically made to the femoral artery of a porcine model. The artery was allowed to bleed out for a period of 45 seconds, following which the haemostatic composition was applied to the bleed site utilising a total combined packing and compression period of 45 seconds. Following the compression period, the wound was assessed for bleeding. If bleeding re-occurred, the haemostatic material compressed for a further one minute pressure. Any re-bleeding after this point was classified as a fail.

The results demonstrated that 66% of the models treated under normal conditions and under coagulopathic conditions obtained haemostasis within the protocol in the femoral artery model within the initial period of 45 seconds. After a further one minute's pressure, 82% of the models treated under normal conditions obtained haemostasis within the protocol in the femoral artery model, whilst 83% of the models treated under coagulopathic conditions obtained haemostasis within the protocol in the femoral artery model.

Under normal conditions the current marketed Celox Rapid haemostat product, requires a protocol of care for 1 minute continuous compression followed by a further 1 minute compression (if required) to achieve haemostasis, whilst in coagulopathic conditions according to recent ISR results Celox Rapid requires 2 minutes continuous compression to achieve haemostasis.

In contrast, the composition of the invention is able to achieve haemostasis a majority of the time—66% under both normal and coagulopathic conditions—in only 45 seconds, and 82% under normal conditions, and 83% under coagulopathic conditions after a further one minute's pressure. This represents a significant improvement, especially in a technical area where the time required to stem the bleeding from a wound is crucial, and can be the difference between life and death for a patient.

It is of course to be understood that the present invention is not intended to be restricted to the foregoing examples which are described by way of example only.

1. A haemostatic composition comprising a haemostat agent, a bioadhesive agent and an antifibrinolytic agent or derivative thereof.

2. A composition according to claim 1, wherein the antifibrinolytic agent comprises one or more selected from tranexamic acid, aminocaproic acid, aminomethylbenzoic acid, aprotinin, epsilon-aminocaproic acid and fibrinogen.

3. A composition according to claim 1, wherein the haemostat agent comprises one or more selected from oxidised regenerated cellulose, kaolin, gelatin, calcium ions, zeolite, collagen, chitosan or a chitosan salt.

4. A composition according to claim 3, wherein the haemostat agent comprises a chitosan salt.

5. A composition according to claim 4, wherein the chitosan salt comprises one or more selected from chitosan acetate, chitosan lactate, chitosan succinate, chitosan malate, chitosan sulphate or chitosan acrylate.

6. A composition according to claim 5, wherein the chitosan salt comprises lactate and/or chitosan succinate.

7. A composition according to claim 1, wherein the bioadhesive agent comprises one or more selected from a carbomer, polyvinyl alcohol (PVA), polyvinylpyrrolidone (PVP), 2-acrylamido-2-methylpropane sulfonic acid, or a high molecular weight acrylic acid polymer cross-linked with divinyl glycol or the salts of polyacrylic acid cross-linked with divinyl glycol.

8. A composition according to claim 7, wherein the bioadhesive agent comprises a cross-linked polymer of acrylic acid, the polymer having a molecular weight of at least about 50,000 g/mol.

9. A composition according to claim 8, wherein the bioadhesive agent comprises one or more selected from: a homopolymer comprising a polymer of acrylic acid cross-linked with allyl sucrose or allyl pentaerythritol; a copolymer comprising a polymer of acrylic acid and C₁₀-C₃₀ alkyl acrylate cross-linked with allyl pentaerythritol; and/or a carbomer homopolymer or copolymer comprising a block copolymer of polyethylene glycol and a long chain alkyl acid ester.

10. A composition according to claim 1, wherein the composition is applied to a carrier material.

11. A composition according to claim 10, wherein the carrier material is selected from a woven material, a non-woven material, a flexible substrate, a film, a foam, or a sheet gel.

12. A haemostatic composition according to claim 1 for use in stemming blood flow from a physiological target site.

13. A method of manufacturing a haemostatic composition according to claim 1, the method comprising the steps of combining a haemostat agent with a bioadhesive agent and an antifibrinolytic agent or derivative thereof.

14. A method according to claim 13, wherein the method comprises the steps of: (1) dispensing a pre-determined weight of a haemostat agent and optionally an inert material into a mixing vessel; (2) dispensing a pre-determined weight of a bioadhesive agent into the mixing vessel containing the haemostat and optional inert material; (3) dispensing a pre-determined weight of an antifibrinolytic agent or derivative thereof; and (4) mixing the haemostat agent, bioadhesive agent and antifibrinolytic agent or derivative thereof.

15. A method of haemostasis, the method comprising the steps of applying the haemostatic composition according to claim 1 to a physiological target site; and applying pressure to the haemostatic material.

16. A method according to claim 15, wherein the pressure is applied for no more than about one minute.

17. A carrier material comprising a haemostatic composition according to claim 1 applied to the carrier material.
